(19) United States
(12) Patent Application Publication
(10) Pub. No.: US 2009/0325890 A1
(43) Pub. Date: Dec. 31, 2009

(54) USE OF GALACTOSE C-GLYCOSIDE DERIVATIVES AS PROTECTIVE AGENT AND/OR GAMA DELTA T LYMPHOCYTE ACTIVATOR

(75) Inventor: Lionel Breton, Versailles (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/296,313

(22) PCT Filed: Apr. 5, 2007
(86) PCT No.: PCT/EP2007/053383

§ 371 Date: Apr. 8, 2009

(30) Foreign Application Priority Data
Apr. 7, 2006 (FR) 0651272

(51) Int. Cl. A61K 31/7028; A61P 43/00; A61K 31/7034

(52) U.S. Cl. 514/23

(57) ABSTRACT

The present invention relates to the use of at least one galactose-derived C-glycoside of general formula (I):

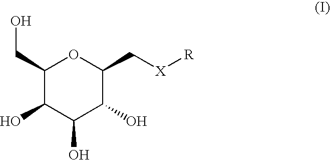

as an agent for protecting and/or stimulating the activity and/or the proliferation of gamma-delta T lymphocyte cells (γδT cells) in a composition containing a cosmetically or pharmaceutically acceptable medium.

The composition will find applications for promoting skin tissue repair, for reequilibrating epidermal proliferation and differentiation disorders which appear with lack of sleep, and for improving the appearance of the head of hair and limiting hair loss.

The present invention relates to the use of at least one galactose-derived C-glycoside of general formula (I):

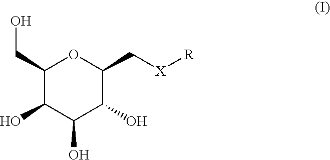

as an agent for protecting and/or stimulating the activity and/or the proliferation of gamma-delta T lymphocyte cells (γδT cells) in a composition containing a cosmetically or pharmaceutically acceptable medium.

The composition will find applications for promoting skin tissue repair, for reequilibrating epidermal proliferation and differentiation disorders which appear with lack of sleep, and for improving the appearance of the head of hair and limiting hair loss.

The human skin consists of a superficial compartment, the epidermis, and a deep compartment, the dermis. The epidermis is mainly composed of three cell types, which are keratinocytes (predominant), melanocytes and Langerhans cells. Each of these cell types contributes by virtue of its own functions to the essential role played in the organism by the skin, in particular the role of protecting the organism against outside attacks. The dermis provides a solid support for the epidermis. It is also its feeder element. It consists mainly of fibroblasts and of an extracellular matrix which is itself composed mainly of collagen, elastin and a ground substance. Leukocytes, mast cells and tissue macrophages are also found therein. Finally, blood vessels and nerve fibres pass through the dermis.

The skin constitutes a barrier against outside attacks, in particular chemical and mechanical attacks, and, in this respect, a certain number of defence reactions against the environment (climate, ultraviolet rays, tobacco, pollutants, etc.) and/or xenobiotics (such as, for example, certain medicaments) take place within it.

In individuals who are in good health, cells damaged by outside attacks are eliminated by the cutaneous immune system. However, for example when an individual experiences a lack of sleep, the immune system is immunodepressed and cannot play its role in monitoring the skin.

Human lymphocytes are characterized by a specific antigen receptor (TCR, T cell receptor). 95% of the lymphocyte population consists of lymphocytes in which the T lymphocyte antigen receptors are transmembrane heterodimers composed of an alpha and beta chain (αβT). Only 5% of lymphocytes express gamma and delta chains (γδT); this population was demonstrated by Tonegawa et al., in 1984.

γδT lymphocytes are mainly located in the blood (for the Vδ2 subtype) and in the epidermis and mucous membranes (for the Vδ1 resident subtype). They express CD3 receptors and gamma-delta TCR receptors (respectively with two variable regions depending on whether they are circulating: Vγ9Vδ2, or resident: Vγ9Vδ1) and are often CD4(−)-CD8(−), their activation is not major histocompatibility complex (MHC)-restricted and the role of cell homeostasis is attributed to them. The gamma chain is encoded by chromosome 7 and the delta chain by 14.

γδT lymphocytes constitute a T lymphocyte population for which the analysis of the structural characteristics has modified the knowledge of interactions between the T cell and its antigen. The importance of the immunoregulatory functions exercised by these cells opens up new perspectives in understanding the mechanisms which contribute to maintaining the homeostasis of the immune system. γδT lymphocytes appear to be involved very early in the regulation of innate immunity.

Recent studies demonstrate that specific T cells play an important role in epidermal homeostasis (Nature Immunology, 6 Jan. 2005, 73-76). In fact, the γδT lymphocytes of the skin and/or the mucous membranes induce the secretion and release of IGF1 in the skin, and the release of IGF1 by keratinocytes results in a control of epidermal homeostasis, in particular the balance between epidermal cell proliferation and differentiation.

The skin undergoes constant remodelling and some of these transformations observed over time are in particular the result of a modification of the natural secretion, by the organism, of hormones (growth hormones, prolactin, oestrogen, etc.) and of growth factors (TGFα and TGFβ, EGF, IGF, etc.).

The role of these hormones and/or of these growth factors is all the more important since, over time, their release and their impact on the target tissues decreases, and tissue growth stabilizes, while the increase in matrix degradation is no longer compensated for by the regenerating action of these hormonal and/or growth factors.

Among these growth factors, IGFs (insulin growth factors) play a predominant role. These factors were discovered while the mechanism of action of growth hormone (GH), which stimulates the growth of all tissues, including skin tissues, was being explored. Growth hormone (GH) consists of 191 amino acids linked in a specific sequence and is secreted by the anterior part of the pituitary gland; this secretion can be reinforced by physical exercise and by other factors. The biological role of GH is fundamental, not only for the growth of a young organism in terms of stature, but also for maintaining its integrity in adulthood. GH acts on the peripheral organs and the brain either directly, or indirectly by stimulating the synthesis of growth factors, such as insulin-like growth factors (IGF I and II) or epidermal growth factor (EGF), or that of their receptors. The direct action of GH is of anti-insulin type by promoting lipolysis in adipose tissues.

By means of IGF1, GH stimulates the incorporation of amino acids into proteins, cartilage and bone growth (statural growth), and cell proliferation in many organs, including the skin.

Growth hormone and the other somatotropic hormones, such as somatomedin C (or IGF1), are capable of maintaining the body's young appearance. They are in fact involved in controlling its metabolism, determining not only the final size of the adult body, but also giving volume, tone and firmness to the organs and tissues, particularly to the muscles. In fact, growth hormone contributes to everything which provides a good self image and a positive mental state. It firms the body and makes the back straighter, and develops the muscles of the shoulders and pelvis. It also decreases fat, especially on the stomach, and increases libido and sexual energy, hair regrowth and colour, and skin elasticity. Perhaps less obviously, but just as beneficially, its effects can be seen in a greater ability to withstand exercise, sleep which is less prolonged and more profitable, an equilibrated arterial blood pressure, and better visual, auditory and cerebral acuity.

In all the mammals studied, including humans, GH is secreted in a pulsatile manner and this characteristic constitutes a determining factor for a large number of biological effects of the hormone.

The causes of the age-related decline in GH are poorly understood. In humans, from puberty, a decrease in GH secretion of the order of 10% every ten years is observed. During ageing, loss of muscle mass, accumulation of adipose tissue, bone demineralization and the loss of tissue regeneration capacity are concomitant with the decrease in GH secretion. The latter promotes an increased catabolism-to-anabolism ratio, thus leading to a situation of imbalance which worsens the effects of ageing.

Additional factors, such as weight gain, or even obesity, related to age, steroid hormone levels, a lack of sleep or a certain degree of tissue resistance to the action of GH can also be important.

In parallel, there is a notable decrease in the quality of sleep with age (decrease in slow wave sleep and in paradoxical sleep and increase in the periods and the duration of awake state which interrupt the phases of sleep). The first phenomenon of ageing is a marked decrease in deep sleep (slow wave sleep or SWS), which can occur as early as the age of 36 and which is replaced by a lighter sleep. The passage from about forty years of age to an elderly age is then associated with a decrease in the amount of sleep and in the duration of the paradoxical phase (rapid eye movement or REM) and of deep sleep (non-REM). Statistics demonstrate that the population affected by sleep disorders comes to 93 million in North America, in Europe and in Japan.

It is conceivable that age-related lack of sleep or qualitative reduction in sleep contributes to hormonal modifications and to the metabolic consequences thereof. In fact, pharmacological treatments which tend to increase slow wave sleep also lead to an increase in GH secretion. Growth hormone deficiency is reflected by physical symptoms: hair loss, fine hair, thin lips and jaw bone, dehydrated skin, sagging stomach, pads of fat at the knees, etc., and psychological symptoms: constant fatigue, difficulties in controlling ones emotions, exhaustion after physical exercise, low self esteem, depression, etc.

The levels of growth hormones circulating in the blood stimulate the production, from the liver, of another hormone, IGF1 (insulin like growth factor 1), the mediator role of which allows growth hormone to develop its positive effects. It is considered to be safer to measure the level of IGF1, also called somatomedin C, than the level of GH, which is virtually undetectable during the day in humans. The liver is the most important site of IGF production, but many cells are capable of producing IGFs. Two types are conventionally described: IGF1 and IGF2. They are two peptides whose amino acid sequence is related to that of insulin, hence their name. Two receptors exist, respectively for IGF1 and for IGF2. The IGF1 receptor has a shared affinity with insulin. This is not the case of the IGF2 receptor.

With ageing, the IGF level decreases and stabilizes in adulthood: this is the somatopause (D. Radman “Effects of human growth hormone in men over 60 years old”: N. Engl J. Med 1990, Juls; 323(1): 1-6).

It is known in the prior art that IGF1 alone stimulates keratinocyte proliferation (Neely E K—Insulin-like growth factors are mitogenic for human keratinocytes and a squamous cell carcimona—J Invest Dermatol 1991 January; 96(1): 104-10). As regards fibroblasts, it is also known that IGF1 stimulates the synthesis of GAGs and the synthesis of collagen. Moreover, studies have demonstrated a not insignificant participation of IGFs in healing. Finally, a study carried out in vivo in humans has demonstrated that percutaneous treatment with IGF1 for one month results in an increase in skin thickness.

It has also been demonstrated that decreased IGF1 expression is associated with increased hair loss (Tang et al., 2003, J. Am. Acad. Dermatol. August; 49(2): 229-33).

The applicant has demonstrated that galactose-derived C-glycoside compounds of general formula (I) stimulate and/or protect γδT cells and that this extract can thus induce the secretion and the release of IGF1 in the skin. This control of IGF1 release by keratinocytes contributes to maintaining epidermal homeostasis, which regulates in particular the balance between epidermal cell proliferation and differentiation.

Thus, according to a first of its subjects, the present invention relates to the use of at least one compound of general formula (I):

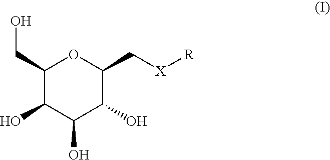

in which,
X represents a group chosen from: —CO—, —CH(NR₁R₂)—, —CHR′— and —C(═CHR′)—;
R represents a linear or branched, saturated or unsaturated alkyl, perfluoroalkyl or hydrofluoroalkyl chain, or a cycloalkyl, cycloperfluoroalkyl or cyclohydrofluoroalkyl ring, containing from 1 to 18 carbon atoms, or a phenyl radical, said chain, said ring or said radical being able to be optionally interrupted with one or more heteroatoms chosen from oxygen, sulphur, nitrogen and silicon, and optionally substituted with at least one radical chosen from —OR′₁, —SR″₁, —NR′″₁R′₂, —COOR″₂, —CONHR′″₂, —CN, halogen, perfluoroalkyl and hydrofluoroalkyl and/or at least one cycloalkyl, aryl or heterocyclic radical, optionally substituted;
R′, v and R₂, which may be identical or different, have the same meaning as that given for R, and can also represent a hydrogen or a hydroxyl radical;
R′₂ and R′″₂, which may be identical or different, represent a hydrogen atom, or a radical chosen from a hydroxyl radical and a linear or branched, saturated or unsaturated alkyl, perfluoroalkyl and/or hydro-fluoroalkyl radical containing from 1 to 20 carbon atoms;
R′₁, R″₁, R″₂ and R′″₁, which may be identical or different, represent a hydrogen atom, or a radical chosen from a linear or branched, saturated or unsaturated alkyl, perfluoroalkyl and/or hydro-fluoroalkyl radical containing from 1 to 20 carbon atoms;
   with the following restrictions:
R′₂ and R′″₁ cannot simultaneously be a hydroxyl;
R₁ and R₂ cannot simultaneously be a hydroxyl radical;
   for maintaining and/or re-establishing the balance between epidermal cell proliferation and differentiation.

Preference will be given to the compounds of general formula (I) as defined above such that:
R represents a linear or branched, saturated or unsaturated alkyl chain, or a cycloalkyl ring, containing from 1 to 10 carbon atoms, or a phenyl radical, said chain, said ring or said radical being capable of being optionally substituted with at least one radical chosen from —OR′₁, —NR′″₁R′₂, —COOR″₂ and —CONHR′″₂;
R′₂ and R′″₂, which may be identical or different, represent a hydrogen atom, or a radical chosen from a hydroxyl radical and a linear or branched, saturated or unsaturated alkyl radical containing from 1 to 8 carbon atoms;
R′₁, R″₁, R″₂ and R′″₁, which may be identical or different, represent a hydrogen atom, or a radical chosen from a linear or branched, saturated or unsaturated alkyl radical containing from 1 to 8 carbon atoms.

More particularly, further preference will be given to the compounds of general formula (I) as defined above, such that:
X represents a group chosen from: —CO—, —CH(NR₁R₂)— and —CHR′;
R represents a linear or branched, saturated or unsaturated alkyl chain, or a cycloalkyl ring, containing from 1 to 10 carbon atoms, or a phenyl radical.

The C-glycoside compounds that can be used according to the invention represent a subfamily of the C-glycoside derivatives described in EP 1 345 919; they can be prepared according to the method described in said document.

Among the C-glycoside derivatives of formula (I) used according to the invention, the following are most particularly preferred:

### Compound 1. 1-(C-β-D-Galactopyranosyl)propan-2-one

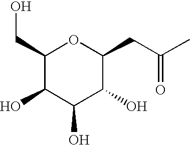

### Compound 2. Phenyl-2-(C-β-D-galactopyranosyl)-1-hydroxyethane

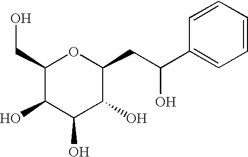

### Compound 3. 1-(C-β-D-Galactopyranosyl)undecan-2-one

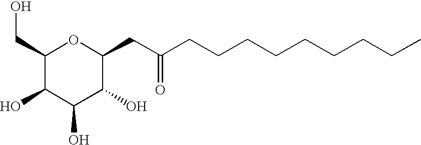

### Compound 4. 1-[2-(3-Hydroxypropylamino)propyl]-C-β-D-galactopyranose

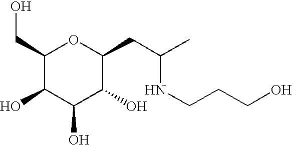

### Compound 5. 3-Methyl-4-(C-β-D-galactopyranosyl)-2-butenoic acid ethyl ester

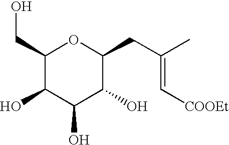

### Compound 6. Ethyl (2E)-3-methyl-4-[(2S,3R,4R,5R,6R)-3,4,5-trihydroxy-6-(hydroxymethyl)tetrahydro-2H-pyran-2-yl]but-2-enoate

The use of an agent for protecting and/or stimulating γδT lymphocytes of the skin and/or the mucous membranes results in increased release of IGF1 by the epidermal cells and promotes physiological proliferation of keratinocytes and/or decreases epidermal differentiation.

Given the fact that IGF1 release is dependent on GH and prolactin secretion and that these hormones are released in a pulsatile manner during the phase of falling asleep and only during the phase of falling asleep (it is a question of a waking state/sleep alternation and not a day/night alternation), the present invention also relates to the cosmetic use of at least one compound of general formula (I), for mimicking the effect of sleep on epidermal cell renewal or for either adding to the effect of sleep on the skin during normal rest or else for supplementing the dysfunction of cutaneous epidermal functions which can appear when there is a lack of sleep.

The use according to the invention will therefore more particularly be aimed at mimicking the effects of sleep on epidermal cell renewal in order to prevent and/or correct the characteristic cutaneous effects of a lack of sleep, and/or at stimulating the skin when there is a lack of sleep and inducing epidermal cell renewal.

Thus, the use according to the invention makes it possible to prevent and/or treat the cutaneous manifestations generated by the slowing down of cell renewal, thus results in cell regeneration in the epidermis and improves the appearance of the surface of the skin.

The consequences of this activity are that the use of the compounds of general formula (I) according to the invention on an individual suffering from lack of sleep makes it possible more particularly to treat drawn and/or sunken features, and to make the complexion uniform.

According to a second subject, the present invention relates to a cosmetic treatment process for effacing the cutaneous signs of fatigue by stimulating epidermal cell regeneration, characterized in that it comprises the application, to the face, of at least one compound of general formula (I).

The use of at least one compound of general formula (I) also makes it possible to prevent and/or treat hair disorders such as a modification of hair density, quantity or quality, a consequence, for example, of a slowing down or an arrest of growth or a loss of hair follicles.

Thus, according to another of its subjects, the present invention relates to the use of at least one compound of general formula (I), for preventing thinning of the keratin fibre and/or inducing growth of the hair and/or of body hairs; for inducing the regrowth of hair or of body hairs which are more dense.

Thus, the invention also relates to the use of at least one compound of general formula (I), as an agent for inducing and/or stimulating the growth of keratin fibres, hair or body hairs, in particular human keratin fibres, hair or body hair, and/or preventing their loss and/or increasing their density.

The expression “increasing the density of keratin fibres, and in particular hair density” is intended to mean increasing the number of keratin fibres, in particular the number of hairs, per cm² of skin such as the scalp.

Thus, another use according to the invention relates to hair treatment compositions (shampoo, lotion, masks, etc.) for limiting and/or preventing hair loss and thus treating alopecia of any nature whatsoever and/or promoting the growth of healthy hair.

The cosmetic use according to the invention of a compound of general formula (I) can be carried out using a cosmetic care and/or makeup composition for keratin fibres.

The invention also applies to keratin fibres of mammals of the animal species (dog, horse or cat, for example).

The human keratin fibres to which the invention applies are in particular the hair, the eyebrows, the eyelashes, beard hair and moustache hair. More especially, the invention applies to human hair and/or eyelashes.

A subject of the present invention is also a cosmetic treatment process for human keratin fibres and/or for the skin from where said fibres emerge, including the scalp, intended in particular to stimulate the growth of human keratin fibres such as the hair and the eyelashes of a human being and/or prevent their loss, characterized in that it consists in applying, to the human keratin fibres and/or the skin from where said fibres emerge, a cosmetic composition comprising an effective amount of at least one compound of general formula (I), in leaving said composition in contact with the keratin fibres and/or the skin from where said fibres emerge, and optionally in rinsing the keratin fibres and/or said skin.

This treatment process has the characteristics of a cosmetic process in so far as it makes it possible to improve the aesthetics of the keratin fibres, and in particular of the hair and the eyelashes, by giving them greater vigour and an improved appearance. In addition, it can be used daily for several months, without medical prescription.

More especially, a subject of the present invention is a cosmetic care process for human hair and/or the human scalp, for the purpose of improving their condition and/or their appearance, characterized in that it consists in applying, to the hair and/or the scalp, a cosmetic composition comprising at least one compound of general formula (I), in leaving said composition in contact with the hair and/or the scalp, and optionally in rinsing the hair and/or the scalp.

A subject of the invention is also a cosmetic care and/or makeup process for human eyelashes, for the purpose of improving their condition and/or their appearance, characterized in that it consists in applying a mascara composition comprising at least one compound of general formula (I) and in leaving said composition in contact with the eyelashes. This mascara composition can be applied alone or as an undercoat for a conventional pigmented mascara and can be removed like a conventional pigmented mascara.

The compositions used according to the invention can be administered orally, enterally or else topically; topical administration will be preferred.

In the case of oral administration, the compositions can be in any suitable form, such as an oral solution, gelatin capsules, sugar-coated tablets, soft or hard capsules, tablets to be swallowed or chewed, granules to be dissolved, a syrup, a solid or liquid food, etc.

The composition may also be in the galenic forms conventionally used for topical application, and in particular in the form of dispersions of the lotion or serum type, emulsions with a liquid or semi-liquid consistency of the milk type, obtained by dispersion of a fatty phase in an aqueous phase (O/W) or conversely (W/O), or suspensions or emulsions with a soft, semi-solid or solid consistency, of the cream or gel type, or alternatively multiple (W/O/W or O/W/O) emulsions, microemulsions, nanoemulsions, vesicular dispersions of ionic and/or non-ionic type, or wax/aqueous phase dispersions. These compositions are prepared to the usual methods.

It may also be in the form of a transdermal system for an active or passive transdermal release of the active agent(s), for example of the patch or gel patch (hydrogel) type.

The composition used according to the invention can thus constitute a treatment or care composition for the skin (including the scalp), keratin fibres (hair, eyelashes, eyebrows), nails or lips, or an antisun or artificial tanning composition, alternatively a cleansing or makeup-removing product for the skin, the hair, the eyebrows or the eyelashes, a deodorant product or a fragrancing compound. It is then generally uncoloured or faintly coloured, and it may optionally contain cosmetic or dermatological active agents. It may then be used as a care base for the skin or the lips (lip balms for protecting the lips against the cold and/or the sun and/or the wind), or as a day or night cream for facial and/or body skin. It may also be in the form of a treating or non-treating, colouring or non-colouring shampoo, or a conditioner.

The composition used according to the invention may also constitute a coloured cosmetic composition, and in particular a makeup composition for the skin, keratin fibres (hair or eyelashes) and/or the mucous membranes, in particular a foundation, a blusher, a face powder, an eyeshadow, a concealer stick, a lipstick or a lip gloss, optionally having care or treating properties. Preferably, it may be a coloured (beige or green) makeup composition for use in correcting the colour of the complexion.

Depending on the purpose of the composition used according to the invention, it may also comprise active agents which will be chosen by those skilled in the art such that they do not harm the effect of the compounds according to the invention.

In the context of the use according to the invention for preventing and/or treating hair disorders, the compositions will be for cosmetic use, and in particular for topical application to the skin and keratin fibres, and more especially to the scalp, the hair and the eyelashes; they may be in any of the known galenic forms suitable for the method of use, for example, those mentioned above.

In particular, the composition for application to the scalp or the hair may be in the form of a hair care lotion, for example for daily or twice-weekly application, a shampoo or a hair conditioner, in particular for twice-weekly or weekly application, a liquid or solid scalp cleaning soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the composition to which the invention applies may be in the form of a pigmented or unpigmented mascara, to be applied to the eyelashes with a brush or alternatively to beard or moustache hair.

According to a specific embodiment, the composition according to the invention is in the form of a hair cream or lotion, a shampoo or hair conditioner, a hair mascara or an eyelash mascara.

According to a specific embodiment of the invention, at least one additional active compound for the hair, that promotes the regrowth and/or that limits the loss of keratin fibres, and in particular of the hair, can be combined with the compound of general formula (I).

Said active agent for the hair may be chosen from:
anti-seborrhoeic agents, such as certain sulphur-containing amino acids, 13-cis-retinoic acid, cyproterone acetate;
agents for combating squamous states of the scalp (dandruff), such as zinc pyrithione, selenium disulphide, climbazole, undecylenic acid, ketoconazole, piroctone olamine (octopirox) or ciclopiroctone (ciclopirox);
active agents that stimulate hair regrowth and/or promote slowing of hair loss; mention may more particularly be made, in a nonlimiting manner, of:
   nicotinic acid esters, including in particular tocopheryl nicotinate, benzyl nicotinate and C₁-C₆ alkyl nicotinates such as methyl nicotinate or hexyl nicotinate;
   pyrimidine derivatives, such as 2,4-diamino-6-piperidinopyrimidine 3-oxide or “Minoxidil” described in U.S. Pat. No. 4,139,619 and U.S. Pat. No. 4,596,812; aminexil or 2,4-diaminopyrimidine 3-oxide described in WO 96/09048;
   agents that are both lipoxygenase inhibitors and cyclooxygenase inducers, or cyclooxygenase-inducing agents that promote hair regrowth, such as those described by the applicant in European patent application EP 0 648 488;
antibiotics such as macrolides, pyranosides and tetracyclines, and in particular erythromycin;
cinnarizine, nimodipine and nifedipine;
hormones, such as estriol or analogues, or thyroxine and salts thereof;
antiandrogenic agents, such as oxendolone, spironolactone, diethylstilbestrol and flutamide;
cromakalim and nicorandil.

The composition according to the invention can be applied to the alopecic areas of the scalp and the hair of an individual, and optionally left in contact for several hours and optionally rinsed off.

### EXAMPLE 1

### Demonstration of the Induction of γδT Lymphocyte Proliferation by the C-Glycoside Derivatives of the Invention

The γδT lymphocyte proliferation activity is tested in the following way: human peripheral blood cells are cultured in the presence of an RPMI-type culture medium supplemented with L-glutamine (2 mM), penicillin/streptomycin (50 μg/50 IU/ml) and foetal calf serum (10%). The C-glycoside derivatives are added at various concentrations (10 to 0.05 mM), along with phytohemagglutin (PHA at 5 *G/ml), a positive control for lymphocyte proliferation. After 3 days of culture, the proliferation is revealed by BrdU labelling.

The results obtained are the following:

| | % stimulation relative | | | | | |
|---|---|---|---|---|---|---|
| Active agent | to the control | | | | | |
| 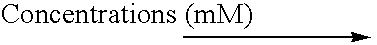 | 10 | 5 | 1 | 0.5 | 0.1 | 0.05 |
| Compound 1: 1-(C-β-D- | 271 | 261 | 138 | 130 | 89 | 91 |
| galactopyranosyl)propan-2- | | | | | | |
| one | | | | | | |

The derivative tested exhibits a high human lymphocyte proliferation capacity at concentrations greater than or equal to 0.5 mM.

### EXAMPLE 2

### Compositions According to the Invention

### Makeup-Removal Lotion for the Face

| | |
|---|---|
| Compound 3 | 1.00 |
| Strontium chloride | 5.00 |
| Antioxidant | 0.05 |
| Isopropanol | 40.00 |
| Preserving agent | 0.30 |
| Water | qs 100%. |

### Facial Care Gel

| | |
|---|---|
| Compound 1 | 5.00 |
| Vichy spring water | 10.00 |
| Thickening polymer | 1.00 |
| Antioxidant | 0.05 |
| Isopropanol | 40.00 |
| Preserving agent | 0.30 |
| Water | qs 100%. |

### “Sleep” Repair Cream

| | |
|---|---|
| Compound 6 | 1.00 |
| Glyceryl stearate | 2.00 |
| Polysorbate 60 (Tween 60 sold by the company ICI) | 1.00 |
| Stearic acid | 1.40 |
| Triethanolamine | 0.70 |
| Carbomer | 0.40 |
| Liquid fraction of shea butter | 12.00 |
| Perhydrosqualene | 12.00 |
| Antioxidant | 0.05 |
| Preserving agent | 0.30 |
| Water | qs 100%. |

### Wax/Water Mascara

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound 5 | 1.00% |
| Black pigment | 5.00% |
| Preserving agent | qs |
| Water | qs 100%. |

This mascara is applied to the eyelashes like a conventional mascara, with a mascara brush.

1. A method for maintaining and/or re-establishing balance between epidermal cell proliferation and differentiation, comprising administering at least one compound of general formula (I): 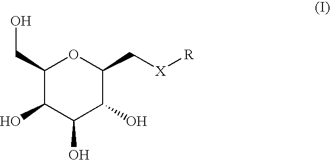
in which,
X represents a —CO—, —CH(NR₁R₂)CHR′— or —C(═CHR′)—;
R represents a linear or branched, saturated or unsaturated alkyl, perfluoroalkyl or hydrofluoroalkyl chain, or a cycloalkyl, cycloperfluoroalkyl or cyclohydrofluoroalkyl ring, containing from 1 to 18 carbon atoms, or a phenyl radical, said chain, said ring or said radical optionally interrupted with one or more heteroatoms selected from the group consisting of oxygen, sulphur, nitrogen and silicon, and optionally substituted with at least one radical selected from the group consisting of —OR′₁, —SR″₁, —NR′″₁R′₂, —COOR″₂, —CONHR′″₂, —CN, halogen, perfluoroalkyl and hydrofluoroalkyl and/or at least one cycloalkyl, aryl or heterocyclic radical, optionally substituted;
R′, R₁ and R₂, which may be identical or different, can have the same meaning as that given for R, and can also represent a hydrogen or a hydroxyl radical;
R′₂ and R′″₂, which may be identical or different, represent a hydrogen atom, or a radical chosen from a hydroxyl radical and a linear or branched, saturated or unsaturated alkyl, perfluoroalkyl and/or hydrofluoroalkyl radical containing from 1 to 20 carbon atoms;
R′₁, R″₁, R″₂ and R′″₁, which may be identical or different, represent a hydrogen atom, or a radical chosen from a linear or branched, saturated or unsaturated alkyl, perfluoroalkyl and/or hydrofluoroalkyl radical containing from 1 to 20 carbon atoms; wherein
R′₂ and R′″₁ cannot simultaneously be a hydroxyl;
R₁ and R₂ cannot simultaneously be a hydroxyl radical;
to skin and/or hair of a subject in need thereof in an amount sufficient for maintaining and/or re-establishing the balance between epidermal cell proliferation and differentiation.

2. The method according to claim 1, wherein:
X represents a group selected from the group consisting of: —CO—, —CH(NR₁R₂)—, —CHR′— and —C(═CHR″)—;
R represents a linear or branched, saturated or unsaturated alkyl, perfluoroalkyl or hydrofluoroalkyl chain, or a cycloalkyl, cycloperfluoroalkyl or cyclohydrofluoroalkyl ring, containing from 1 to 14 carbon atoms, or a phenyl or benzyl radical, said chain, said ring or said radical optionally interrupted with one or more heteroatoms chosen from oxygen, sulphur, nitrogen and silicon, and optionally substituted with at least one radical chosen from —OR′₁, —SR″₁, —NR₁′R′₂, —COOR″₂, —CONHR₂′, —CN, halogen, perfluoroalkyl and hydrofluoroalkyl and/or at least one cycloalkyl, aryl or heterocyclic radical, optionally substituted;
R′, R₁ and R₂, which may be identical or different, can have the same meaning as R, and can also represent a hydrogen or a hydroxyl radical;
R″ can have the same definition as R, and can also represent a hydroxyl radical;
R′₂ and R₂′, which may be identical or different, represent a hydrogen atom, or a radical chosen from a hydroxyl radical and a linear or branched, saturated or unsaturated alkyl, perfluoroalkyl and/or hydrofluoroalkyl radical containing from 1 to 14 carbon atoms;
R′₁, R″₁, R″₂ and R₁′, which may be identical or different, represent a hydrogen atom, or a radical chosen from a linear or branched, saturated or unsaturated alkyl, perfluoroalkyl and/or hydrofluoroalkyl radical containing from 1 to 14 carbon atoms;
wherein (i) R₁ and R₂ cannot simultaneously be a hydroxyl radical, (ii) R′₂ and R₁′ cannot simultaneously be a hydroxyl radical and (iii) when R is an alkyl with 2 or 3 carbon atoms, then R cannot be substituted with a —COOR″₂ group.

3. The method according to claim 1, wherein:
R represents a linear or branched, saturated or unsaturated alkyl chain, or a cycloalkyl ring, containing from 1 to 10 carbon atoms, or a phenyl radical, said chain, said ring or said radical optionally substituted with at least one radical chosen from —R′₂ and R₂′, which may be identical or different, represent a hydrogen atom, or a radical chosen from a hydroxyl radical and a linear or branched, saturated or unsaturated alkyl radical containing from 1 to 8 carbon atoms;
R′₁, R″₁, R″₂ and R₁′, which may be identical or different, represent a hydrogen atom, or a radical chosen from a linear or branched, saturated or unsaturated alkyl radical containing from 1 to 8 carbon atoms.

4. The method according to claim 1, wherein:
X represents a group chosen from: —CO—, —CH(NR₁R₂)— and —CHR′;
R represents a linear or branched, saturated or unsaturated alkyl chain, or a cycloalkyl ring, containing from 1 to 10 carbon atoms, or a phenyl radical.

5. The method according to claim 1, wherein the compound of general formula (I) is chosen from:
Compound 1. 1-(C-β-D-Galactopyranosyl)propan-2-one;
Compound 2. Phenyl-2-(C-β-D-galactopyranosyl)-1-hydroxyethane;
Compound 3. 1-(C-β-D-Galactopyranosyl)undecan-2-one;
Compound 4. 1-[2-(3-Hydroxypropylamino)propyl]-C-β-D-galactopyranose;
Compound 5. 3-Methyl-4-(C-β-D-galactopyranosyl)-2-butenoic acid ethyl ester; and
Compound 6. Ethyl (2E)-3-methyl-4-[(2S,3R,4R,5R,6R)-3,4,5-trihydroxy-6-(hydroxymethyl)tetrahydro-2H-pyran-2-yl]but-2-enoate.

6. The method according to claim 1, wherein the administration of the compound of formula (I) mimics the effects of sleep on epidermal cell renewal.

7. The method according to claim 6, wherein the administration of the compound of formula (I) corrects the cutaneous effects caused by lack of sleep.

8. The method according to claim 6, wherein the administration of the compound of formula (I) induces epidermal cell renewal.

9. The method according to claim 6, wherein the administration of the compound of formula (I) induces epidermal cell regeneration.

10. The method according to claim 6, wherein the administration of the compound of formula (I) improves the appearance of the surface of the skin and/or treating drawn or sunken features and/or making the complexion uniform.

11. The method according to claim 1, wherein the administration of the compound of formula (I) reduces thinning of the keratin fiber of the hair and/or inducing growth of the hair and/or of body hairs.

12. The method according to claim 11, wherein the administration of the compound of formula (I) induces regrowth of the hair or of body hairs which are more dense.

13. The method according to claim 1, wherein the administration of the compound of formula (I) treats hair loss and/or the loss of body hairs.

14. The method according to claim 13, f wherein the administration of the compound of formula (I) treats alopecia.

15. The method according to claim 1, which further comprises administering with the at least one compound of general formula (I) is combined with at least one active agent chosen from agents for treating hair loss and/or agents for activating regrowth of the hair and of body hairs.

16. A cosmetic treatment process for effacing cutaneous signs of fatigue by stimulating epidermal cell regeneration, the method comprising applying to the face at least one compound of general formula (I) 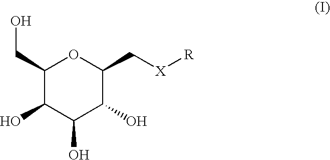
in which,
X represents a —CO—, —CH(NR₁R₂)CHR′— or —C(═CHR′)—;
R represents a linear or branched, saturated or unsaturated alkyl, perfluoroalkyl or hydrofluoroalkyl chain, or a cycloalkyl, cycloperfluoroalkyl or cyclohydrofluoroalkyl ring, containing from 1 to 18 carbon atoms, or a phenyl radical, said chain, said ring or said radical optionally interrupted with one or more heteroatoms selected from the group consisting of oxygen, sulphur, nitrogen and silicon, and optionally substituted with at least one radical selected from the group consisting of —OR′₁, —SR″₁, —NR′″₁R′₂, —COOR″₂, —CONHR′″₂, —CN, halogen, perfluoroalkyl and hydrofluoroalkyl and/or at least one cycloalkyl, aryl or heterocyclic radical, optionally substituted;
R′, R₁ and R₂, which may be identical or different, can have the same meaning as that given for R, and can also represent a hydrogen or a hydroxyl radical;
R′₂ and R′″₂, which may be identical or different, represent a hydrogen atom, or a radical chosen from a hydroxyl radical and a linear or branched, saturated or unsaturated alkyl, perfluoroalkyl and/or hydrofluoroalkyl radical containing from 1 to 20 carbon atoms;
R′₁, R″₁, R″₂ and R′″₁, which may be identical or different, represent a hydrogen atom, or a radical chosen from a linear or branched, saturated or unsaturated alkyl, perfluoroalkyl and/or hydrofluoroalkyl radical containing from 1 to 20 carbon atoms; wherein
R′₂ and R′″₁ cannot simultaneously be a hydroxyl;
R₁ and R₂ cannot simultaneously be a hydroxyl radical.

17. A cosmetic treatment process for stimulating growth of human keratin fibres and/or reduce loss of keratin fibres,
the method comprising applying, to human keratin fibres and/or skin from where said fibres emerge, a cosmetic composition comprising at least one compound of general formula (I) 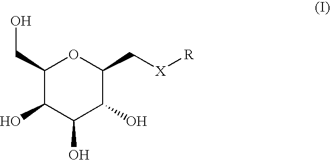
in which,
X represents a —CO—, —CH(NR₁R₂)CHR′— or —C(═CHR′)—;
R represents a linear or branched, saturated or unsaturated alkyl, perfluoroalkyl or hydrofluoroalkyl chain, or a cycloalkyl, cycloperfluoroalkyl or cyclohydrofluoroalkyl ring, containing from 1 to 18 carbon atoms, or a phenyl radical, said chain, said ring or said radical optionally interrupted with one or more heteroatoms selected from the group consisting of oxygen, sulphur, nitrogen and silicon, and optionally substituted with at least one radical selected from the group consisting of —OR′₁, —SR″₁, —NR′″₁R′₂, —COOR″₂, —CONHR′″₂, —CN, halogen, perfluoroalkyl and hydrofluoroalkyl and/or at least one cycloalkyl, aryl or heterocyclic radical, optionally substituted;
R′, R₁ and R₂, which may be identical or different, can have the same meaning as that given for R, and can also represent a hydrogen or a hydroxyl radical;
R′₂ and R′″₂, which may be identical or different, represent a hydrogen atom, or a radical chosen from a hydroxyl radical and a linear or branched, saturated or unsaturated alkyl, perfluoroalkyl and/or hydrofluoroalkyl radical containing from 1 to 20 carbon atoms;
R′₁, R″₁, R″₂ and R′″₁, which may be identical or different, represent a hydrogen atom, or a radical chosen from a linear or branched, saturated or unsaturated alkyl, perfluoroalkyl and/or hydrofluoroalkyl radical containing from 1 to 20 carbon atoms; wherein
R′₂ and R′″₁ cannot simultaneously be a hydroxyl;
R₁ and R₂ cannot simultaneously be a hydroxyl radical,
leaving said composition in contact with the keratin fibres and/or the skin from where said fibres emerge, and
OPTIONALLY, RINSING THE KERATIN FIBRES AND/OR SAID SKIN.
